(19) United States
(12) Patent Application Publication
(10) Pub. No.: US 2009/0081285 A1
(43) Pub. Date: Mar. 26, 2009

(54) USE OF RADICAL-CAPTURING SUBSTANCES IN A TOPICAL PREPARATION FOR ANTIPYRETIC TREATMENT

(76) Inventors: Karin Golz-Berner, Monaco (MC); Leonhard Zastrow, Monaco (MC)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/913,488

(22) PCT Filed: May 4, 2006
(86) PCT No.: PCT/EP2006/062075

§ 371 Date: Jun. 30, 2008

(30) Foreign Application Priority Data
May 4, 2005 (DE) 10 2005 021 806.7

(51) Int. Cl. A61K 9/48; A61K 38/44; A61K 9/10; A61K 9/28; A61K 9/20

(52) U.S. Cl. 424/451; 424/94.4; 424/484; 424/464; 424/474

(57) ABSTRACT

The present invention relates to the use of one or more radical-capturing substances as therapeutically active substances in a topical preparation for antipyretic treatment. The invention is also directed to the use of radical-capturing substances as active agents in a combined preparation for the treatment of fever, said combined preparation comprising a topical preparation and an oral preparation in free combination, and the two preparations independently including one or more free-radical-capturing substances.

According to the invention, the two preparations of the combined preparation are intended for simultaneous, separate or time-differentiated administration.

The present invention relates to the use of one or more radical-capturing substances as therapeutically active substances in a topical preparation for antipyretic treatment. The invention is also directed to the use of radical-capturing substances in the form of active agents in a combined preparation for the treatment of fever, said combined preparation comprising a topical preparation and an oral preparation in free combination, and the two preparations independently including one or more free-radical-capturing substances. According to the invention, the two preparations of the combined preparation are intended for simultaneous, separate or time-differentiated administration.

Fever is a physiological condition characterized by an increased body temperature. A level of 38-38.5° C. is referred to as moderate fever, 39-40.5° C. as high fever, and more than that as very high fever.

At present, two classes of active substances find use as oral antipyretic substances, namely, the salicylates and p-aminophenol derivatives. The salicylates, characterized by acetylsalicylic acid (for example, aspirin), are the most widely used antipyretic active substances. Although aspirin is well-tolerated by most humans in principle, a number of toxic side effects are associated with the use thereof, namely, salicylate-induced gastric ulcer and sometimes gastrointestinal hemorrhage. The p-aminophenol derivatives acetaminophen and phenacetin are alternatives to aspirin with respect to their antipyretic effect, with acetaminophen having a somewhat lower toxicity compared to phenacetin and, in addition, lacks the undesirable side effects of aspirin. However, acetaminophen involves the risk of hepatic necrosis in cases of acute overdosing. Chronic overdosing may give rise to hemolytic anemia as one form of acute toxicity.

The object of the invention was to find alternative active substances for the treatment of fever.

According to the invention, said object is accomplished by providing a topically applicable preparation which includes radical-capturing substances or mixtures of substances.

It was found that an increase of the skin temperature above 37° C., especially in the range of 37 to 45° C., results in a rapid increase of free radicals on the skin, and that formation of free radicals as a result of body temperature increase not only can be reduced by means of radical scavengers, particularly antioxidants, which have a specific capacity of capturing free radicals, but also an antipyretic effect is induced at the same time.

In particular, suitable according to the invention are therefore those topical preparations which have a radical protection factor of at least 250×10¹⁴ radicals per mg of preparation, measured by determining the number of free radicals of a solution of test substance (S₁) by means of electron spin resonance (ESR) and comparing with the ESR measured result of the preparation according to the relationship:

RPF=(RC×RF)/PI,

wherein RF=(S₁−S₂)/S₁; RC=concentration of test substance (radicals/ml); PI=concentration of active substance preparation (mg/ml); S₂=signal amplitude of a solution of antioxidant. The radical protection factor is preferably 800×10¹⁴ radicals per mg of preparation, more preferably 30,000×10¹⁴ radicals per mg of preparation.

The radical protection factor (RPF) indicates the activity in binding free radicals by an antioxidant or radical scavenger compared to a test substance.

The radical scavenger(s) can be applied to the skin e.g. in a dermatological preparation.

In addition to the radical scavenger(s), the preparations of the invention include dermatological adjuvants and excipients, as usually employed in such preparations, e.g. water, preservatives, dyes, thickeners, humectants, alcohols, polyols, esters, electrolytes, gelling agents, polar and non-polar oils, polymers, copolymers, emulsifiers, stabilizers and fillers.

For topical application of the radical-capturing substances, they are formulated in a usual manner together with at least one pharmaceutically acceptable carrier and optionally further adjuvants to form solid formulations applicable to the skin, such as creams, gels, ointments or emulsions, or liquid formulations applicable to the skin, such as solutions, suspensions, lotions, douches, serums or oils.

Suitable bases for ointments, creams or gels are, for example, vaseline, paraffins such as hard paraffin or viscous paraffin, medium-chain triglycerides, natural waxes, wool wax, isopropyl myristate, highly dispersed silica, bentonite, starch, alginates, cellulose and cellulose ethers, sodium carboxymethylcellulose, polyethylene glycols, etc.

Suitable solvents for lotions and solutions are water or water-alcohol mixtures.

As will be appreciated, liposomes, cyclodextrins or nanoparticles ensuring optimum transport of antioxidants into the skin can also be used as carrier systems for the antioxidants. Also possible as topical preparations are transdermal systems, e.g. adhesives, patches or dressings, which include the antioxidants together with a carrier.

To support passage of the antioxidants through the skin, useful carriers can contain absorbable, pharmacologically suitable solvents.

For example, solvents ensuring good penetration of the radical-capturing substances into the skin are the alcohols phenylethanol-1, glycerol or ethanol or mixtures thereof.

In a preferred embodiment of the invention the topical preparations include stabilizers for the antioxidants.

Also, advantageous therapeutic preparations are aqueous systems in the form of tinctures or baths, or dry substances intended for use in the preparation of a bath.

According to the invention, any commonly known enzymatic and non-enzymatic antioxidants can be used as radical-capturing substances as long as they can be formulated into a dermally applicable preparation and exhibit an appropriate radical protection factor.

For example, the antioxidants used are selected from the group of vitamins consisting of tocopherols and derivatives thereof, especially α-tocopherol or α-tocopheryl esters, particularly tocopheryl acetate, tocopheryl acylate, laurate, myristate, palmitate, oleate or linoleate; vitamin A and derivatives thereof, particularly retinyl palmitate; vitamin C and derivatives thereof, particularly isoascorbate, 2- or 3- or 6-O-alkylascorbic acids, ascorbic esters such as ascorbyl acetates, ascorbyl phosphates, 6-O-lauroyl-, myristoyl-, palmitoyl-, oleoyl- or linoleoyl-L-ascorbic acid; folic acid and derivatives thereof.

Other radical scavengers usable according to the invention are selected from the group consisting of flavonoids comprising flavones, flavonols, flavanonals and chacones, especially citrus flavonoids such as rutin, naringin and neohesperidin; carotenoids and carotenes such as α-carotene and β-carotene; α-lipoic acid, lipoic amide; amino acids such as histidine, glycine, tyrosine, tryptophan and amino acid derivatives; α-hydroxy acids such as citric acid, lactic acid, malic acid, uric acid and derivatives thereof; rutinic acid, α-glucosylrutin; phenolcarboxylic acids such as rosmarinic acid or ferulic acid; humic acid; bile acid and bile acid derivatives such as methyl, ethyl, propyl, amyl, butyl and lauryl gallate; bile extracts; unsaturated fatty acids; ubiquinone, ubiquinol; zinc and salts thereof; selenium compounds; coenzyme Q10; urocaninic acid; lecithin; anthocyans; polyphenols; tetrahydrodiferuloylmethane (THC).

The preparations from plant extracts with a high radical protection factor, referred to as RPF complex and described in WO 99/66881, WO 01/26617 and DE 103 25 156 A1, can also be used as antioxidants in the present invention.

Other advantageous plant extracts are acerola extract, extracts of citrus peels or leaves (Citrus bigaradia, Citrus hystrix, Citrus aurantifolia, Citrofurtunella micro-carpa, Citrus aurantium, Citrus reticulata), bitter orange extract (peels or fruit), cherry extract from Spanish cherry, kiwi extract (Actinidia quinensis), papaya fruit extract (Caricae papayae), tea extract [leaves of green or black tea, leaves or bark of New Jersey tea (Ceanthus velutinas)], coffee bean extract of green or roasted beans, prunus extracts, e.g. from Prunus armeniaca, Prunus dulcis, Prunus persica, Prunus domestica, Prunus spinosa, Prunus serotina, Prunus virginiana, bark extracts of the Mexican skin tree (Mimosa tenuiflora), angelica root extract (Angelica archangelica), Pongamia pinnata extract, tomato extract.

The content of the above plant extracts in the topical preparation can be between 0.05 and 45 wt.-%, preferably 0.1 to 40 wt.-%, especially 1.5 to 20 wt.-%, and mixtures of these extracts can also be included in the active substance preparation. The concentration depends on the radical protection factor of the extract or radical scavenger. Thus, extracts having very high radical protection factors of 10,000 to 90,000 can be included at relatively low concentrations of 0.1 wt.-%, provided they roughly retain the appropriate radical protection factor for a prolonged period of time of several weeks to months.

Specifically preferred are radical scavenger contents of 3 to 33 wt.-%, particularly 12 to 26 wt.-%, relative to the overall weight of the topical preparation.

The radical protection factor of the preparation is at least 300×10¹⁴ radicals per mg of preparation, particularly at least 800×10¹⁴ radicals per mg, and especially preferably at least 1,400×10¹⁴ radicals per mg.

Specifically preferred are embodiments of the invention wherein the radical protection factor of the preparation is 1,500 to 30,000×10¹⁴ radicals per mg of preparation.

The concentration of the radical scavenger or mixture or radical scavengers is preferably in the range of 5 to 40 wt.-%, particularly in the range of 8 to 35 wt.-%, relative to the overall weight of the preparation.

According to the invention, it is also possible to use enzymatic antioxidants such as superoxide dismutase and metal complexes having similar activity, such as catalase and glutathione peroxidase.

Another preferred radical scavenger is the above-mentioned RPF complex I from WO 99/66881 (e.g. Example 1 or 2) or WO 01/26617 which consists of an active substance preparation having a content of a product obtained by extraction of the bark of Quebracho blanco and subsequent enzymatic hydrolysis, which product includes at least 90 wt.-% of proanthocyanidin oligomers and at most 10 wt.-% of gallic acid, in microcapsules, and a silkworm extract obtained by extraction, which includes the peptide cecropin, amino acids and a vitamin mixture, and a non-ionic, cationic or anionic hydrogel or a mixture of hydrogels, and one or more phospholipids and water (RPF 2400), optionally supplemented by cyclo-dextrins and a yeast decomposition product (RPF 4800) described below.

Also, an advantageous radical scavenger is a mixture of enzymes and vitamins, specifically a yeast decomposition product produced by ultrasonication, said decomposition product containing SOD, protease, vitamin B₂, vitamin B₆, vitamin B₁₂, vitamin D₂ and vitamin E. In a preferred fashion it contains at least 150 U/ml SOD, protease and the vitamins B and D, the ratio of SOD/protease as international units being at least in the range of from 3:1 to 8:1 (RPF 2020×10¹⁴ radicals/mg). The enzyme/vitamin mixture is produced via a decomposition process using ultrasound, which has been described in DE 42 411 54 C1 and wherein a cell dispersion or suspension is passed through a sonication space in an ultra-sound flow cell, the sonotrode extending over the half to two-thirds of its length into the flow cell, immersing into the medium to be sonicated. The sonotrode has an angle of 80.5 to 88.5°, and the ratio of immersed length of the sonotrode in mm to sonication volume in ml is adjusted to a value of 1:1.1 to 1:20. The solids content in the medium to be sonicated ranges from 1:0.02 to 1:2.2 (wt.-%). Yeasts such as baker's yeast, brewer's yeast, wine yeast as well as specially treated yeasts, such as SOD-enriched yeasts, can be used as cell dispersion. A cell dispersion used with advantage contains e.g. Saccharomyces cerevisiae.

Addition of e.g. 1-10 wt.-% of such a yeast decomposition product of baker's yeast or bioyeast can augment an existing radical protection factor of another oxidant in a synergistic fashion.

Other preferred radical scavengers include (RPF values in brackets, with no added “×10¹⁴ radicals/mg”) tomato extract (1,000); carrot extract (300); RPF complex+vitamin E in cyclodextrin (7,200); stabilized vitamin C (8290); ultra-sound-treated yeast decomposition product from baker's yeast (2020); rape extract (67,000); RPF complex I in cyclodextrins (720); oregano oil (Origanox) (90,306); Origanum vulgare extract (80,000); tannic acid (310,000); pine bark extract (12,500); Himothatus sucruba extract (700); Emplica® (Merck) (42,400); white grape peel (53,000); red grape peel (95,100); flavonoid extract from red wine (6,000); rosmarinic acid (36,000-68,000); curry extract (12,500); saffron extract (900); orange peel extract (24,000); rape oil (2,550); strawberry oil (1,300); green tea extract (21,500); grapefruit extract (53,000); sodium ascorbyl phosphate (35,000); edelweiss extract (15,500); Camellia sinensis extract (840).

In an advantageous embodiment of the invention, the topical preparation includes the radical scavenger or mixture of radical scavengers encapsulated in usual liposomes or in asymmetrical lamellar aggregates. These aggregates are constituted of phospholipids and oxygen-loaded fluorocarbon or a mixture of fluorocarbons. Their content of fluorocarbon is in the range of from 0.2 to 100% weight/volume, the phospholipid having a phosphatidyl choline content of preferably more than 30 to 99 wt.-%, and said aggregates, depending on the critical solubility temperature of the fluorocarbon, enable penetration into the skin. The aggregates are oxygen carriers and enable oxygen penetration into the skin and thus improved oxygen supply of the skin. The preparation may also include aggregates bearing oxygen only, thereby further supporting the effect of the radical scavenger.

These aggregates are produced using high-pressure homogenization of phospholipids, such as soy lecithin and egg lecithin, or synthetic phospholipids or partially hydrogenated phospholipids having a phosphatidyl choline content of more than 30 to 99 wt.-% with perfluorinated or highly fluorinated carbon compounds or mixtures thereof that are capable of transporting gases such as oxygen and carbon dioxide. In addition to phosphatidyl choline, lysolecithins in a concentration range of from 0.1 to 10 wt.-% and/or charged phospholipids such as phosphatidyl ethanolamine, phosphatidyl N-acetylethanolamine or phosphatidic acid in a concentration range of from 0.1 to 30 wt.-%, relative to the overall weight of the aggregates, can be present therein.

Unlike well-known aqueous liposomes (vesicles), these phospholipid-stabilized aggregates carry in their core hydrophobic fluorocarbons capable of transporting oxygen. The interfacial chemical stabilization thereof is primarily effected by a monolayer with inverse arrangement and possibly a subsequent structure of bi-layers. Owing to the peculiarity of their structural arrangement, these aggregates are referred to as asymmetrical lamellar oxygen carriers. Their exceptional colloid-chemical stability is presumably based on the lamellar structure and the surface charge of the aggregates. The latter is to be attributed to the selection of suitable phospholipids or their mixtures of natural and synthetic origin. Above all, it is the phospholipids that are responsible for an advantageous effect in the meaning above, and particularly phosphatidyl choline. The above-mentioned effect of the phospholipids is verified by corresponding negative zeta potentials and by measuring the charge densities (using titration with a cationic polyelectrolyte). What is essential to the use of fluorocarbon aggregates is skin penetration depending on the critical solubility temperature of the selected fluorocarbons or mixture of fluorocarbons (for the use of asymmetrical lamellar aggregates see also DE-B-42 21 255).

The term “fluorocarbons” used here is understood as meaning perfluorinated or highly fluorinated carbon compounds or mixtures which are able to transport gases such as oxygen and carbon dioxide. Highly fluorinated hydrocarbon compounds within the meaning of this invention are those in which most of the hydrogen atoms are replaced by fluorine atoms, so that on further replacement the capability for gas transport is not necessarily increased. This is usually achieved if approximately up to 90% of the hydrogen atoms have been replaced by fluorine atoms. Preferred fluorocarbons in the meaning of the present invention are those in which at least 95% of the hydrogen atoms are replaced, more preferably 98% and most preferably 100%. A variety of fluorocarbons can be employed, e.g. aliphatic straight-chain and branched fluoroalkanes, mono- or bicyclic and optionally fluoroalkyl-substituted fluorocycloalkanes, perfluorinated aliphatic or bicyclic amines, bis(perfluoroalkyl)ethenes, perfluoropolyethers or mixtures thereof. Particularly preferred fluorocarbons are those such as perfluorodecalin, F-butyltetrahydrofuran, perfluorotributylamine, perfluorooctyl bromide, bis-fluoro(butyl)ethene or bis-fluoro(hexyl)ethene or C₆-C₉ perfluoroalkanes.

The topical preparation of the invention may also include humectants such as glycerol, butylene glycol, propylene glycol or mixtures thereof.

As set forth above, usual liposomes can be used as transport system for the modified kaolin-containing mixture in the preparation of the invention. Liposomes are completely closed lipid bilayer membranes containing an entrapped aqueous volume. Liposomes may be unilamellar vesicles (having a single bilayer membrane) or multilamellar vesicles (onion-like structures characterized by multiple membrane bilayers, each separated from the next by an aqueous layer). The bi-layer is constituted of two lipid monolayers having a hydrophobic “tail” region and a hydrophilic “head” region. The structure of the membrane bilayer is such that the hydrophobic (non-polar) “tails” of the lipid monolayers orient toward the center of the bilayer, while the hydrophilic “heads” orient towards the aqueous phase. The production of liposomes from saturated and unsaturated lipids as well as the use thereof as transport system has been described in a large number of patents.

For example, phosphatidyl choline, phosphatidyl ethanolamine, phosphatidyl inositol, phosphatidyl serine, phosphatidic acid and lysolecithins as well as mixtures thereof are employed as phospholipids. Well-known products are Phos-lipon® or Nat®, for example.

The oils used in the preparation of the invention can be conventional cosmetic oils, such as mineral oil; hydrogenated polyisobutene; squalane produced synthetically or from natural products; cosmetic esters or ethers which can be branched or unbranched, saturated or unsaturated; vegetable oils; or mixtures of two or more thereof. Especially suitable oils are, for example, silicone oils, mineral oils, hydrogenated polyisobutene, polyisoprene, squalanes, tridecyl trimellitate, trimethylpropane triisostearate, isodecyl citrate, neopentyl glycol diheptanoate, PPG-15 stearyl ethers as well as vegetable oils, such as calendula oil, jojoba oil, avocado oil, macadamia nut oil, castor oil, cocoa butter, coconut oil, maize oil, cottonseed oil, olive oil, palm nut oil, rapeseed oil, safflower oil, sesame oil, soybean oil, sunflower oil, wheat germ oil, grape seed oil, candlenut oil, thistle oil and mixtures thereof.

Depending on which oils are selected, the dermatological properties of a solid composition are affected, such as degree of transparency, softness, hardness and spreading effect.

The preparations of the invention can be in the form of O/W or W/O emulsions. Suitable emulsifiers for O/W emulsions are e.g. addition products of 2-30 moles of ethylene oxide with linear C₈-C₂₂ fatty alcohols, with C₁₂-C₂₂ fatty acids and with C₈-C₁₅ alkylphenols; C₁₂-C₂₂ fatty acid mono- and diesters of addition products of 1-30 moles of ethylene oxide with glycerol.

Suitable emulsifiers for W/O emulsions are, for example, addition products of 2-15 moles of ethylene oxide with castor oil; esters of C₁₂-C2₂ fatty acids and glycerol, polyglycerol, pentaerythritol, sugar alcohols (e.g. sorbitol), polyglucosides (e.g. cellulose); polyalkylene glycols; wool wax alcohols; copolymers of poly-siloxane-polyalkyl polyethers.

As set forth above, the radical protection factor (RPF) determines the activity of a substance for binding free radicals as compared with a test substance. The test substance consists of a highly reactive, semi-stable radical which reacts with all known antioxidants. Such radicals include nitroxides such as Proxo (2,2,5,5-tetramethyl-1-dihydropyrrolinoxy nitroxide), Tempol (2,2,6,6-tetramethyl-1-piperidinoxy-4-ol nitroxide), DTBN (di-tert-butyl-nitroxide) or preferably DPPH (1,1-diphenyl-2-picryl-hydrazyl).

The RPF is measured in such a way that the signal amplitude of the test radical is measured by electron spin resonance (ESR/EPR) before and after mixing with an antioxidant/radical scavenger and calculating the RPF therefrom. For a series of standard antioxidants the RPF is well-known. Thus, it is 827 for all-trans-retinol, 196 for all-trans-retinol acetate, 41,200 for DL-α-tocopherol and about 48 for α-tocopheryl acetate, each time×10¹⁴ radicals/mg.

The exact method for measuring the radical protection factor has been described by Herrling, Groth, Fuchs and Zastrow in Conference Materials “Modern Challenges to the Cosmetic Formulation” 5.5.-7-5.97, Düsseldorf, pp. 150-155, Verlag f. chem. Ind. 1997. Starting from the known concentration of test substance (here: DPPH) or the number of its free radicals (radicals per ml), a signal amplitude S₁ is measured using an ESR spectrometer. Both test radical and antioxidant are dissolved in a water/alcohol solution (e.g. 0.1 M). Thereafter, the signal amplitude S₂ of the antioxidant is measured. The normalized difference between the two signal amplitudes is the reduction factor RF.

RF=(S₁−S₂)/S₁

The result of the radical reduction of the test substance RC×RF is normalized relative to the quantity of product input PI (mg/ml). RC is the amount of the test substance, i.e., the known number of radicals in the test substance. The radical protection factor is calculated by means of the following equation:

$$R P F = \frac{R C [ radicals \text{/} ml ] \times R F}{P I [ mg \text{/} ml ]}$$

The result is

RPF=N×10¹⁴[radicals per mg],

wherein N is a positive real number and RPF for simplification may be reduced to the numerical value of N. This reduction will be used in the examples of the present invention.

The radical protection factor can be determined using an ESR spectrometer (GALENUS GmbH, Berlin, Germany) and is a magnitude for characterizing products with respect to their capability of binding free radicals. The method is an in vitro method in which no individual properties of the user would influence the antioxidants.

Surprisingly, a further increase of this factor by 1.3 to 10 times is observed when adding cyclodextrins which have a radical protection factor of 0.

Commercially available α-, β- or γ-cyclodextrins (Wacker Chemie) or mixtures thereof can be employed as cyclodextrins. Cyclodextrins are well-known as encapsulating materials for pharmaceutical and cosmetic active substances and can therefore be used for the encapsulation of radical scavengers as well.

The above-mentioned topical preparations include the radical-capturing substances in a therapeutically effective amount. This amount may depend on various factors such as sex, age, and individual condition of a patient, as well as the severity of the fever. In a preferred embodiment of the invention the final preparation includes 5-45 wt.-% antioxidant, preferably 5-40 wt.-%, more preferably 10-35 wt.-%, especially 10-20 wt.-%, relative to the overall weight of the preparation.

Typically, the topical pharmaceutical preparation, e.g. in the form of a gel or cream, is applied 2 to 6 times a day, allowing a time of 1 to 2 hours after the first application before repeating. In a preferred fashion the topical preparation is applied at intervals of one hour. The application preferably can be effected on the calves, arms and/or on the back. In general, high fever of 39-40.5° C. is reduced by 1-3° C. within a few hours and after 1 to 2 days at the latest.

It will be clear that the suggested topical treatment can also be used as a supplement in an oral antipyretic treatment. Therefore, the present invention is also directed to the above-described use of the above-mentioned radical-capturing substances in combination with simultaneous oral administration of well-known antipyretic agents, in which case the dosage of such antipyretic agents can be reduced by 30-70%.

Hence, the invention also relates to a method for the antipyretic treatment, which method is characterized in that a therapeutic preparation comprising a therapeutically effective amount of a radical-capturing substance or a mixture of radical-capturing substances is applied to the skin of a human in a preferred quantity of at least 2 mg/cm², more preferably 2-10 mg/cm². More specifically, the topical preparation is applied to the arms, legs and/or back, preferably for at least 4 hours and up to 2 days. The radical protection factor of the preparation should be at least 250×10¹⁴ radicals per mg of preparation.

Simultaneous administration of oral antipyretic agents is possible in such a method. In a preferred fashion the administered dose of oral antipyretic agents is reduced by 30-70%.

The invention is also directed to the use of radical-capturing substances as therapeutically active substances in the production of a combined preparation for antipyretic treatment, which comprises a freely combined topical preparation and an oral preparation, each of said preparations comprising one or more radical-capturing substances. The invention is also directed to the combined preparation itself.

It was found that fever can be controlled even more effectively by simultaneous or sequential systemic and topical administration of radical-capturing substances.

According to the invention, the oral preparation includes 5 to 20 wt.-%, particularly 5 to 15 wt.-% (relative to the overall weight of the oral preparation) radical-capturing substance(s). The radical-capturing substances of the oral preparation are selected from the group of vitamins consisting of tocopherols and derivatives thereof, especially α-tocopherol; vitamin A and derivatives thereof, particularly retinyl palmitate; vitamin C and derivatives thereof, particularly isoascorbate, 2- or 3- or 6-O-alkylascorbic acids, ascorbic esters such as ascorbyl acetates, ascorbyl phosphates, 6-O-lauroyl-, myristoyl-, palmitoyl-, oleoyl- or linoleoyl-L-ascorbic acid; folic acid and derivatives thereof. The radical-capturing substances of the oral preparation can also be selected from the group consisting of flavonoids comprising flavones, flavonols, flavanonals and chacones, especially citrus flavonoids such as rutin, naringin and neohesperidin; carotenoids and carotenes such as α-carotene and β-carotene; α-lipoic acid, lipoic amide; amino acids such as histidine, glycine, tyrosine, tryptophan and amino acid derivatives; α-hydroxy acids such as citric acid, lactic acid, malic acid; rutinic acid, α-glucosylrutin; phenolcarboxylic acids such as rosmarinic acid or ferulic acid.

For example, tablets, film tablets, coated tablets, capsules, pills, powders, solutions, or suspensions, including depot forms, are used as oral preparation of the combined preparation of the invention.

Medicinal drugs in the form of tablets can be obtained for example by mixing the radical scavenger with well-known adjuvants such as dextrose, sugar, sorbitol, mannitol, polyvinylpyrrolidone, disintegrants such as corn starch or alginic acid, binders such as starch or gelatin, lubricants such as magnesium stearate or talc, and/or agents which can achieve a depot effect, such as carboxy-polymethylene, carboxymethylcellulose, cellulose acetate phthalate or polyvinyl acetate. The tablets may also be constituted of several layers.

Similarly, coated tablets can be prepared by coating cores, produced in a similar way as tablets, with agents conventionally used in tablet coatings, e.g. polyvinylpyrrolidone or shellac, gum arabic, talc, titanium dioxide, or sugar. The tablet coating may also be constituted of several layers, to which end the above-mentioned adjuvants are used.

Capsules can be prepared by mixing the active substance with vehicles such as lactose or sorbitol which are subsequently incorporated in the capsules.

To improve the taste, the solutions, dispersions or suspensions including the radical scavenger can be mixed with materials such as saccharin, cyclamate or some types of sugars and/or with flavoring agents such as vanillin or orange extract. Moreover, they can be mixed with suspending aids such as sodium carboxymethylcellulose or with preservatives such as p-hydroxybenzoic acid.

The topical preparation of the free combination includes the above-mentioned radical-capturing substances in amounts likewise mentioned above.

Consequently, the invention is also directed to a method for the antipyretic treatment of humans, wherein a topical preparation comprising a therapeutically effective amount of one or more radical-capturing substances is applied to the skin of a human, with simultaneous or sequential application of an oral preparation comprising a therapeutically effective amount of one or more radical-capturing substances.

In a preferred fashion the oral preparation, e.g. a tablet, should be administered simultaneously with the beginning of topical application, e.g. in the morning, and optionally repeated at noon and/or in the evening, preferably at least in the evening. The daily oral dose of radical scavenger(s) for an adult is between 20 and 170 mg, preferably 100 to 150 mg. This dose can be distributed over 2 to 3 administrations per day.

Without intending to be limiting, the invention will be explained in more detail with reference to the following examples.

### EXAMPLE 1

### Antipyretic cream I

Phase A

| | |
|---|---|
| Isopropyl myristate | 3.0 |
| Steareth-2 | 2.3 |
| Steareth-21 | 1.5 |
| PPG-15 stearyl ether | 3.0 |

Phase B

| Water | q.s. ad 100 |
|---|---|
| EDTA | 0.04 |
| Carbomers | 0.3 |
| Water/NaOH | 0.3 |
| Glycerol | 2.0 |

Phase C

| | |
|---|---|
| Dimethicone | 2.0 |

Phase D

| | |
|---|---|
| Preservative | 0.1 |
| Menthol | 1.0 |

Phase E

| | |
|---|---|
| RPF complex¹ | 10.0 |
| Red grape peel extract | 1.0 |
| Rosmarinic acid | 0.5 |
| Origanox ® WS | 1.0 |
| Tannic acid | 1.0 |

¹in accordance with WO 99/66881 (active substance complex according to Example 1).

The phases A and B, separately produced, are heated to 75° C. and combined with stirring. The mixture is cooled to approximately 45° C., and phase C is added with stirring. This is subsequently cooled to 40° C. Following addition of phase D with stirring, phase E is finally added at 35° C., and the mixture is stirred to be homogeneous.

### RPF=4,990×10¹⁴ rad./mg.

### EXAMPLE 2

### Antipyretic cream II

Phase A

| | |
|---|---|
| Isopropyl myristate | 3.0 |
| Steareth-2 | 2.3 |
| Steareth-21 | 1.5 |
| PPG-15 stearyl ether | 3.0 |

Phase B

| Water | q.s. ad 100 |
|---|---|
| EDTA | 0.04 |
| Carbomers | 0.3 |
| Water/NaOH | 0.3 |
| Glycerol | 2.0 |

Phase C

| | |
|---|---|
| Dimethicone | 2.0 |

Phase D

| | |
|---|---|
| Preservative | 0.1 |
| Menthol | 1.0 |

Phase E

| | |
|---|---|
| Vitamin C, stabilized | 9.5 |
| White grape peel extract | 1.3 |
| Rosmarinic acid | 0.6 |
| Tannic acid | 1.0 |
| Green Tee extract | 0.7 |

### EXAMPLE 3

The cream of Example 1 was applied to the arms and legs of a non-medicated male aged 62 and suffering from an antipyretic drug allergy. The body temperature of this male dropped from 39.4° C. to 38.5° C. after 2 hours and to 38.1° C. after another 2 hours. No skin irritation appeared.

### EXAMPLE 4

The cream of Example 1 was applied to the arms, legs and back of a female 26 years of age 24 hours after discontinuing treatment with an antipyretic agent. The female's body temperature dropped from 39.7° C. to 39.0° C. after 3 hours and to 38.6° C. after another 3 hours. No skin irritation appeared.

### EXAMPLE 5

The cream of Example 2 was applied to the arms, legs and back of a female 38 years of age. The female's body temperature dropped from 39.1° C. to 38.4° C. after 4 hours and to 37.9° C. after another 3 hours. No skin irritation appeared.

### EXAMPLE 6

The cream of Example 2 was applied hourly to the arms, legs and back of seven subjects suffering from fever between 39.0° C. and 39.4° C. After three hours the fever had dropped by 0.5-0.6° C. and by another 0.3-0.4° C. after another two hours.

### EXAMPLE 7

### Vitamin Tablet

A tablet having the following composition of radical scavengers was produced: vitamin C 98 mg, vitamin E 22 mg, niacin 15 mg, pantothenic acid B₅ 2 mg, β-carotene 7 mg, vitamin B₆ 1.7 mg, vitamin B₂ 1.4 mg, folic acid 0.3 mg, vitamin B₁ 1.1 mg, vitamin B₁₂ 3 mg, adjuvants: microcrystalline cellulose, magnesium stearate, sorbitol and corn starch.

### EXAMPLE 8

### Combined Therapy

The cream of Example 1 was applied hourly to the arms, legs and back of ten subjects suffering from fever between 39.2° C. and 39.4° C. In a parallel measure, each one of five subjects were administered with one tablet according to Example 7 at the beginning of topical application. The other five subjects did not receive a tablet.

In the former five subjects who had received topical and oral application in parallel, the fever had dropped to 38.2-38.4° C. after only three hours. After another two hours of hourly cream application, the body temperature of these subjects was 37.7-38° C.

The five subjects who had received hourly topical application of the cream of Example 1 showed a body temperature lowered by 0.5-0.7° C. after three hours. After another two hours of hourly cream application to arms, legs and back, their body temperature was 38.2-38.5° C.

1-20. (canceled)

21. Use of one or more radical-capturing substances as therapeutically active substances for the production of a topical preparation for antipyretic treatment, wherein the radical-capturing substances are selected from the group consisting of a RPF complex being a mixture of plant extracts on alcoholic basis which consists of 0.1 to 2% by weight extract from green coffee beans, 0.1 to 2% by weight extract from the leaves of Camellia sinensis, 0.1 to 2% by weight extract from Pongamia pinnata and 0.1 to 2% by weight extract from the roots of Angelica archangelica and a monovalent C₂-C₅ alcohol making up the remainder up to 100% by weight,
a RPF complex which comprises a content of
a) a product obtained by extraction of the bark of Quebracho blanco and subsequent enzymatic hydrolysis containing at least 90 percent by weight of proanthocyanidine oligomers and up to 10 percent by weight of gallic acid, where the content of (a), which is available in a concentration of 2 percent by weight linked to a microcapsule, ranges from 0.1 to 10 wt. %;
(b) a silkworm extract obtained by extraction containing the peptide cecropin, amino acids and a vitamin mixture, where the content of (b) ranges from 0.1 to 10 wt. %;
(c) a non-ionic, cationic or anionic hydrogel or a mixture of hydrogels, where the content of (c) ranges from 0.1 to 5 wt %;
(d) one or more phospholipids in the range of 0.1 to 30 wt. %;
(e) water,
and a RPF complex which comprises a content of
a) a product obtained by extraction of the bark of Quebracho blanco and subsequent enzymatic hydrolysis, containing at least 90 percent by weight of proanthocyanidine oligomers and up to 10 percent by weight of gallic acid, where the content of (a), which is available in a concentration of 2 percent by weight linked to a microcapsule ranges from 0.1 to 10 wt. %;
(b) a silkworm extract obtained by extraction containing the peptide cecropin, amino acids and a vitamin mixture, where the content of (b) ranges from 0.1 to 10 wt. %;
(c) a non-ionic, cationic or anionic hydrogel or a mixture of hydrogels, where the content of (c) ranges from 0.1 to 5 wt %;
(d) one or more phospholipids in the range of 0.1 to 30 wt. %;
(e) an ultrasound decomposition product of a yeast containing at least 150 units of superoxide dismutase per ml, wherein the content of the decomposition product is in the range from 0.5 to 4 percent by weight;
(f) one or more cyclodextrines selected from ∝, β- and γ-cyclodextrins with a content of 0.5 to 8% by weight; and
(g) the remainder being water;
related to the total weight of the RPF complex each,
mixtures thereof and mixtures with radical-capturing substances selected from the group consisting of flavonoids comprising flavones, flavonols, flavanonals and chacones, especially citrus flavonoids such as rutin, naringin and neohesperidin; carotenoids and carotenes such as α-carotene and β-carotene; α-lipoic acid, lipoic amide; amino acids such as histidine, glycine, tyrosine, tryptophan and amino acid derivatives; α-hydroxy acids such as citric acid, lactic acid, malic acid; uric acid and derivatives thereof; rutinic acid, α-glucosylrutin; phenolcarboxylic acids such as rosmarinic acid or ferulic acid; humic acid; bile acid and bile acid derivatives such as methyl, ethyl, propyl, amyl, butyl and lauryl gallate; bile extracts; unsaturated fatty acids; ubiquinone, ubiquinol; zinc and salts thereof; selenium compounds; coenzyme Q10; urocaninic acid; lecithin; anthocyans; polyphenols; tetrahydrodiferuloylmethane (THC).

22. The use according to claim 21,
characterized in that
the radical protection factor of the preparation is at least 250×10¹⁴ radicals per mg of preparation, measured by determining the number of free radicals of a solution of test substance (S₁) by means of electron spin resonance (ESR) and comparing with the ESR measured result of the preparation according to the relationship:
RPF=(RC×RF)/PI,
wherein RF=(S₁−S₂)/S₁; RC=concentration of test substance (radicals/ml); PI=concentration of active substance preparation (mg/ml); S₂=signal amplitude of a solution of antioxidant,
preferably 800×10¹⁴ radicals/mg preparation, particularly 30,000×10¹⁴ radicals/mg preparation.

23. The use according to claim 21,
characterized in that
the topical preparation includes 5-45 wt.-% radical-capturing substance(s), preferably 10-20 wt.-%.

24. The use according to claim 21,
characterized in that
the topical preparation is applied in the form of a cream, ointment, lotion, tincture, bath, in liquid or solid form, douche, serum, gel or oil.

25. Use of radical-capturing substances as therapeutically active substances in the production of a combined preparation for antipyretic treatment, which comprises a topical preparation and an oral preparation in free combination, each of said preparations comprising one or more radical-capturing substances and the radical-capturing substances of the topical preparation being selected from the group consisting of a RPF complex
being a mixture of plant extracts on alcoholic basis which consists of 0.1 to 2% by weight extract from green coffee beans, 0.1 to 2% by weight extract from the leaves of Camellia sinensis, 0.1 to 2% by weight extract from Pongamia pinnata and 0.1 to 2% by weight extract from the roots of Angelica archangelica and a monovalent C₂-C₅ alcohol making up the remainder up to 100% by weight,
a RPF complex which comprises a content of
a) a product obtained by extraction of the bark of Quebracho blanco and subsequent enzymatic hydrolysis containing at least 90 percent by weight of proanthocyanidine oligomers and up to 10 percent by weight of gallic acid, where the content of (a), which is available in a concentration of 2 percent by weight linked to a microcapsule, ranges from 0.1 to 10 wt. %;
(b) a silkworm extract obtained by extraction containing the peptide cecropin, amino acids and a vitamin mixture, where the content of (b) ranges from 0.1 to 10 wt. %;
(c) a non-ionic, cationic or anionic hydrogel or a mixture of hydrogels, where the content of (c) ranges from 0.1 to 5 wt %;
(d) one or more phospholipids in the range of 0.1 to 30 wt. %;
(e) water,
and a RPF complex which comprises a content of
a) a product obtained by extraction of the bark of Quebracho blanco and subsequent enzymatic hydrolysis, containing at least 90 percent by weight of proanthocyanidine oligomers and up to 10 percent by weight of gallic acid, where the content of (a), which is available in a concentration of 2 percent by weight linked to a microcapsule ranges from 0.1 to 10 wt. %;
(b) a silkworm extract obtained by extraction containing the peptide cecropin, amino acids and a vitamin mixture, where the content of (b) ranges from 0.1 to 10 wt. %;
(c) a non-ionic, cationic or anionic hydrogel or a mixture of hydrogels, where the content of (c) ranges from 0.1 to 5 wt %;
(d) one or more phospholipids in the range of 0.1 to 30 wt. %;
(e) an ultrasound decomposition product of a yeast containing at least 150 units of superoxide dismutase per ml, wherein the content of the decomposition product is in the range from 0.5 to 4 percent by weight;
(f) one or more cyclodextrines selected from ∝, β- and γ-cyclodextrins with a content of 0.5 to 8% by weight; and
(g) the remainder being water;
related to the total weight of the RPF complex each,
mixtures thereof and mixtures with radical-capturing substances selected from the group consisting of flavonoids comprising flavones, flavonols, flavanonals and chacones, especially citrus flavonoids such as rutin, naringin and neohesperidin; carotenoids and carotenes such as α-carotene and β-carotene; α-lipoic acid, lipoic amide; amino acids such as histidine, glycine, tyrosine, tryptophan and amino acid derivatives; α-hydroxy acids such as citric acid, lactic acid, malic acid; uric acid and derivatives thereof; rutinic acid, α-glucosylrutin; phenolcarboxylic acids such as rosmarinic acid or ferulic acid; humic acid; bile acid and bile acid derivatives such as methyl, ethyl, propyl, amyl, butyl and lauryl gallate; bile extracts; unsaturated fatty acids; ubiquinone, ubiquinol; zinc and salts thereof; selenium compounds; coenzyme Q10; urocaninic acid; lecithin; anthocyans; polyphenols; tetrahydrodiferuloylmethane (THC).

26. The use according to claim 25,
characterized in that
the oral preparation, relative to the overall weight of the oral preparation, includes 5-20 wt.-%, preferably 5-15 wt.-% radical-capturing substances.

27. The use according to claim 25,
characterized in that
the radical-capturing substances of the oral preparation are selected from the group of vitamins consisting of tocopherols and derivatives thereof, especially α-tocopherol; vitamin A and derivatives thereof, particularly retinyl palmitate; vitamin C and derivatives thereof, particularly isoascorbate, 2- or 3- or 6-O-alkylascorbic acids, ascorbic esters such as ascorbyl acetates, ascorbyl phosphates, 6-O-lauroyl-, myristoyl-, palmitoyl-, oleoyl- or linoleoyl-L-ascorbic acid; folic acid and derivatives and mixtures thereof.

28. The use according to claim 25,
characterized in that
the radical-capturing substances of the oral preparation are selected from the group consisting of flavonoids comprising flavones, flavonols, flavanonals and chacones, especially citrus flavonoids such as rutin, naringin and neohesperidin; carotenoids and carotenes such as α-carotene and β-carotene; α-lipoic acid, lipoic amide; amino acids such as histidine, glycine, tyrosine, tryptophan and amino acid derivatives; α-hydroxy acids such as citric acid, lactic acid, malic acid; rutinic acid, α-glucosylrutin; phenolcarboxylic acids such as rosmarinic acid or ferulic acid.

29. The use according to claim 25,
characterized in that
the topical preparation includes 5-45 wt.-% radical-capturing substances, preferably 10-20 wt.-%.

30. The use according to claim 25,
characterized in that
the topical preparation and the oral preparation are administered simultaneously or sequentially.

31. The use according to claim 25,
characterized in that
a tablet, capsule, coated tablet, powder, solution or suspension is employed as oral preparation.

32. A method for the antipyretic treatment of humans,
characterized in that
a topical preparation comprising a therapeutically effective amount of one or more radical-capturing substances is applied to the skin of a human, particularly to arms, legs and/or back, preferably at hourly intervals, wherein the radical-capturing substances are selected from the group consisting of a RPF complex
being a mixture of plant extracts on alcoholic basis which consists of 0.1 to 2% by weight extract from green coffee beans, 0.1 to 2% by weight extract from the leaves of Camellia sinensis, 0.1 to 2% by weight extract from Pongamia pinnata and 0.1 to 2% by weight extract from the roots of Angelica archangelica and a monovalent C₂-C₅ alcohol making up the remainder up to 100% by weight,
a RPF complex which comprises a content of
a) a product obtained by extraction of the bark of Quebracho blanco and subsequent enzymatic hydrolysis containing at least 90 percent by weight of proanthocyanidine oligomers and up to 10 percent by weight of gallic acid, where the content of (a), which is available in a concentration of 2 percent by weight linked to a microcapsule, ranges from 0.1 to 10 wt. %;
(b) a silkworm extract obtained by extraction containing the peptide cecropin, amino acids and a vitamin mixture, where the content of (b) ranges from 0.1 to 10 wt. %;
(c) a non-ionic, cationic or anionic hydrogel or a mixture of hydrogels, where the content of (c) ranges from 0.1 to 5 wt %;
(d) one or more phospholipids in the range of 0.1 to 30 wt. %;
(e) water,
and a RPF complex which comprises a content of
a) a product obtained by extraction of the bark of Quebracho blanco and subsequent enzymatic hydrolysis, containing at least 90 percent by weight of proanthocyanidine oligomers and up to 10 percent by weight of gallic acid, where the content of (a), which is available in a concentration of 2 percent by weight linked to a microcapsule ranges from 0.1 to 10 wt. %;
(b) a silkworm extract obtained by extraction containing the peptide cecropin, amino acids and a vitamin mixture, where the content of (b) ranges from 0.1 to 10 wt. %;
(c) a non-ionic, cationic or anionic hydrogel or a mixture of hydrogels, where the content of (c) ranges from 0.1 to 5 wt %;
(d) one or more phospholipids in the range of 0.1 to 30 wt. %;
(e) an ultrasound decomposition product of a yeast containing at least 150 units of superoxide dismutase per ml, wherein the content of the decomposition product is in the range from 0.5 to 4 percent by weight;
(f) one or more cyclodextrines selected from ∝, β- and γ-cyclodextrins with a content of 0.5 to 8% by weight; and
(g) the remainder being water;
related to the total weight of the RPF complex each,
mixtures thereof and mixtures with radical-capturing substances selected from the group consisting of flavonoids comprising flavones, flavonols, flavanonals and chacones, especially citrus flavonoids such as rutin, naringin and neohesperidin; carotenoids and carotenes such as α-carotene and β-carotene; α-lipoic acid, lipoic amide; amino acids such as histidine, glycine, tyrosine, tryptophan and amino acid derivatives; α-hydroxy acids such as citric acid, lactic acid, malic acid; uric acid and derivatives thereof; rutinic acid, α-glucosylrutin; phenolcarboxylic acids such as rosmarinic acid or ferulic acid; humic acid; bile acid and bile acid derivatives such as methyl, ethyl, propyl, amyl, butyl and lauryl gallate; bile extracts; unsaturated fatty acids; ubiquinone, ubiquinol; zinc and salts thereof; selenium compounds; coenzyme Q10; urocaninic acid; lecithin; anthocyans; polyphenols; tetrahydrodiferuloylmethane (THC).

33. The method according to claim 32,
characterized in that
the radical protection factor of the preparation is at least 250×10¹⁴ radicals per mg of preparation, measured by determining the number of free radicals of a solution of test substance (S₁) by means of electron spin resonance (ESR) and comparing with the ESR measured result of the preparation according to the relationship:
RPF=(RC×RF)/PI,
wherein RF=(S₁−S₂)/S₁; RC=concentration of test substance (radicals/ml); PI=concentration of active substance preparation (mg/ml); S₂=signal amplitude of a solution of antioxidant.

34. The method according to claim 32,
characterized in that
application of the radical-capturing substance(s) and an oral administration of an antipyretic agent are effected in parallel, the dose of the latter being 30-70% lower than usual.

35. A method for the antipyretic treatment of humans,
characterized in that
a topical preparation comprising a therapeutically effective amount of one or more radical-capturing substances is applied to the skin of a human, wherein the radical-capturing substances are selected from the group consisting of a RPF complex being a mixture of plant extracts on alcoholic basis which consists of 0.1 to 2% by weight extract from green coffee beans, 0.1 to 2% by weight extract from the leaves of Camellia sinensis, 0.1 to 2% by weight extract from Pongamia pinnata and 0.1 to 2% by weight extract from the roots of Angelica archangelica and a monovalent C₂-C₅ alcohol making up the remainder up to 100% by weight,
a RPF complex which comprises a content of
a) a product obtained by extraction of the bark of Quebracho blanco and subsequent enzymatic hydrolysis containing at least 90 percent by weight of proanthocyanidine oligomers and up to 10 percent by weight of gallic acid, where the content of (a), which is available in a concentration of 2 percent by weight linked to a microcapsule, ranges from 0.1 to 10 wt. %;
(b) a silkworm extract obtained by extraction containing the peptide cecropin, amino acids and a vitamin mixture, where the content of (b) ranges from 0.1 to 10 wt. %;
(c) a non-ionic, cationic or anionic hydrogel or a mixture of hydrogels, where the content of (c) ranges from 0.1 to 5 wt %;
(d) one or more phospholipids in the range of 0.1 to 30 wt. %;
(e) water,
and a RPF complex which comprises a content of
a) a product obtained by extraction of the bark of Quebracho blanco and subsequent enzymatic hydrolysis, containing at least 90 percent by weight of proanthocyanidine oligomers and up to 10 percent by weight of gallic acid, where the content of (a), which is available in a concentration of 2 percent by weight linked to a microcapsule ranges from 0.1 to 10 wt. %;
(b) a silkworm extract obtained by extraction containing the peptide cecropin, amino acids and a vitamin mixture, where the content of (b) ranges from 0.1 to 10 wt. %;
(c) a non-ionic, cationic or anionic hydrogel or a mixture of hydrogels, where the content of (c) ranges from 0.1 to 5 wt %;
(d) one or more phospholipids in the range of 0.1 to 30 wt. %;
(e) an ultrasound decomposition product of a yeast containing at least 150 units of superoxide dismutase per ml, wherein the content of the decomposition product is in the range from 0.5 to 4 percent by weight;
(f) one or more cyclodextrines selected from ∝, β- and γ-cyclodextrins with a content of 0.5 to 8% by weight; and
(g) the remainder being water;
related to the total weight of the RPF complex each,
mixtures thereof and mixtures with radical-capturing substances selected from the group consisting of flavonoids comprising flavones, flavonols, flavanonals and chacones, especially citrus flavonoids such as rutin, naringin and neohesperidin; carotenoids and carotenes such as α-carotene and β-carotene; α-lipoic acid, lipoic amide; amino acids such as histidine, glycine, tyrosine, tryptophan and amino acid derivatives; α-hydroxy acids such as citric acid, lactic acid, malic acid; uric acid and derivatives thereof; rutinic acid, α-glucosylrutin; phenolcarboxylic acids such as rosmarinic acid or ferulic acid; humic acid; bile acid and bile acid derivatives such as methyl, ethyl, propyl, amyl, butyl and lauryl gallate; bile extracts; unsaturated fatty acids; ubiquinone, ubiquinol; zinc and salts thereof; selenium compounds; coenzyme Q10; urocaninic acid; lecithin; anthocyans; polyphenols; tetrahydrodiferuloylmethane (THC)
and an oral preparation independently comprising a therapeutically effective amount of one or more radical-capturing substances is applied simultaneously or sequentially.

36. A combined preparation for antipyretic treatment, comprising a topical and an oral preparation in free combination, the two preparations independently comprising a therapeutically effective amount of one or more radical-capturing substances and the radical-capturing substances of the topical preparation being selected from the group consisting of a RPF complex
being a mixture of plant extracts on alcoholic basis which consists of 0.1 to 2% by weight extract from green coffee beans, 0.1 to 2% by weight extract from the leaves of Camellia sinensis, 0.1 to 2% by weight extract from Pongamia pinnata and 0.1 to 2% by weight extract from the roots of Angelica archangelica and a monovalent C₂-C₅ alcohol making up the remainder up to 100% by weight,
a RPF complex which comprises a content of
a) a product obtained by extraction of the bark of Quebracho blanco and subsequent enzymatic hydrolysis containing at least 90 percent by weight of proanthocyanidine oligomers and up to 10 percent by weight of gallic acid, where the content of (a), which is available in a concentration of 2 percent by weight linked to a microcapsule, ranges from 0.1 to 10 wt. %;
(b) a silkworm extract obtained by extraction containing the peptide cecropin, amino acids and a vitamin mixture, where the content of (b) ranges from 0.1 to 10 wt. %;
(c) a non-ionic, cationic or anionic hydrogel or a mixture of hydrogels, where the content of (c) ranges from 0.1 to 5 wt %;
(d) one or more phospholipids in the range of 0.1 to 30 wt. %;
(e) water,
and a RPF complex which comprises a content of
a) a product obtained by extraction of the bark of Quebracho blanco and subsequent enzymatic hydrolysis, containing at least 90 percent by weight of proanthocyanidine oligomers and up to 10 percent by weight of gallic acid, where the content of (a), which is available in a concentration of 2 percent by weight linked to a microcapsule ranges from 0.1 to 10 wt. %;
(b) a silkworm extract obtained by extraction containing the peptide cecropin, amino acids and a vitamin mixture, where the content of (b) ranges from 0.1 to 10 wt. %;
(c) a non-ionic, cationic or anionic hydrogel or a mixture of hydrogels, where the content of (c) ranges from 0.1 to 5 wt %;
(d) one or more phospholipids in the range of 0.1 to 30 wt. %;
(e) an ultrasound decomposition product of a yeast containing at least 150 units of superoxide dismutase per ml, wherein the content of the decomposition product is in the range from 0.5 to 4 percent by weight;
(f) one or more cyclodextrines selected from ∝, β- and γ-cyclodextrins with a content of 0.5 to 8% by weight; and
(g) the remainder being water;
related to the total weight of the RPF complex each,
mixtures thereof and mixtures with radical-capturing substances selected from the group consisting of flavonoids comprising flavones, flavonols, flavanonals and chacones, especially citrus flavonoids such as rutin, naringin and neohesperidin; carotenoids and carotenes such as α-carotene and β-carotene; α-lipoic acid, lipoic amide; amino acids such as histidine, glycine, tyrosine, tryptophan and amino acid derivatives; α-hydroxy acids such as citric acid, lactic acid, malic acid; uric acid and derivatives thereof; rutinic acid, α-glucosylrutin; phenolcarboxylic acids such as rosmarinic acid or ferulic acid; humic acid; bile acid and bile acid derivatives such as methyl, ethyl, propyl, amyl, butyl and lauryl gallate; bile extracts; unsaturated fatty acids; ubiquinone, ubiquinol; zinc and salts thereof; selenium compounds; coenzyme Q10; urocaninic acid; lecithin; anthocyans; polyphenols; tetrahydrodiferuloylmethane (THC).
